SEQUENCE LISTING (24 sequences)

SEQ ID NO: 1 - DNA, 351 bp; single-stranded, linear
  CDS 1..351
caggtgcagctggtggagtctgggggaggcgttgtgcagcctggaaggtccctgagactctcctgtgcagcctctggattcaccttcagtagctatgacatgtcttgggttcgccaggctccgggcaagggtctggagtgggtcgcaaaagttagtagtggtggtggtagcacctactatttagacactgtgcagggccgattcaccatctccagagacaatagtaagaacaccctatacctgcaaatgaactctctgagagccgaggacacagccgtgtattactgtgcaagacataactacggcagttttgcttactggggccaagggactacagtgactgtttctagt

SEQ ID NO: 2 - protein, 117 aa
QVQLVESGGGVVQPGRSLRLSCAASGFTFSSYDMSWVRQAPGKGLEWVAKVSSGGGSTYYLDTVQGRFTISRDNSKNTLYLQMNSLRAEDTAVYYCARHNYGSFAYWGQGTTVTVSS

SEQ ID NO: 3 - DNA, 321 bp; single-stranded, linear
  CDS 1..321
gagattgtgctaactcagtctccagccaccctgtctctcagcccaggagaaagggcgactctttcctgccaggccagccaaagtattagcaaccacctacactggtatcaacaaaggcctggtcaagccccaaggcttctcatcmkktatcgttcccagtccatctctgggatccccgccaggttcagtggcagtggatcagggacagatttcaccctcactatctccagtctggagcctgaagattttgcagtctattactgtcaacagagtggcagctggcctcacacgttcggaggggggaccaaggtggaaattaag

SEQ ID NO: 4 - protein, 107 aa
EIVLTQSPATLSLSPGERATLSCQASQSISNHLHWYQQRPGQAPRLLIXYRSQSISGIPARFSGSGSGTDFTLTISSLEPEDFAVYYCQQSGSWPHTFGGGTKVEIK

SEQ ID NO: 5 - DNA, 351 bp; single-stranded, linear
  CDS 1..351
gaagtgcagctggtggagtctgggggaggcttagtgaagcctggaaggtccctgagactctcctgtgcagcctctggattcgctttcagtagctatgacatgtcttgggttcgccagattccggagaagaggctggagtgggtcgcaaaagttagtagtggtggtggtagcacctactatttagacactgtgcagggccgattcaccatctccagagacaatgccaagaacaccctatacctgcaaatgagcagtctgaactctgaggacacagccatgtattactgtgcaagacataactacggcagttttgcttactggggccaagggactctggtcactgtctctgca

SEQ ID NO: 6 - protein, 117 aa
EVQLVESGGGLVKPGRSLRLSCAASGFAFSSYDMSWVRQIPEKRLEWVAKVSSGGGSTYYLDTVQGRFTISRDNAKNTLYLQMSSLNSEDTAMYYCARHNYGSFAYWGQGTLVTVSA

SEQ ID NO: 7 - DNA, 321 bp; single-stranded, linear
  CDS 1..321
gatattgtgctaactcagtctccagccaccctgtctgtgacaccaggagatagcgtcagtctttcctgccaggccagccaaagtattagcaaccacctacactggtatcaacaaaaatcacatgagtctccaaggcttctcatcaagtatcgttcccagtccatctctgggatcccctccaggttcagtggcagtggatcagggacagatttcgctctcagtatcaacagtgtggagactgaagattttggaatgtatttctgtcaacagagtggcagctggcctcacacgttcggaggggggaccaagctggaaattaag

SEQ ID NO: 8 - protein, 107 aa
DIVLTQSPATLSVTPGDSVSLSCQASQSISNHLHWYQQKSHESPRLLIKYRSQSISGIPSRFSGSGSGTDFALSINSVETEDFGMYFCQQSGSWPHTFGGGTKLEIK

SEQ ID NO: 9 - DNA, 84 bp; single-stranded, linear
caggtgcagctggtggagtctgggggaggcgttgtgcagcctggaaggtccctgagactctcctgtgcagcctctggattcacc

SEQ ID NO: 10 - DNA, 84 bp; single-stranded, linear
aacttttgcgacccactccagacccttgcccggagcctggcgaacccaagacatgtcatagctactgaaggtgaatccagaggc

SEQ ID NO: 11 - DNA, 87 bp; single-stranded, linear
tgggtcgcaaaagttagtagtggtggtggtagcacctactatttagacactgtgcagggccgattcaccatctccagagacaatagt

SEQ ID NO: 12 - DNA, 81 bp; single-stranded, linear
tgcacagtaatacacggctgtgtcctcggctctcagagagttcatttgcaggtatagggtgttcttactattgtctctgga

SEQ ID NO: 13 - DNA, 75 bp; single-stranded, linear
gtgtattactgtgcaagacataactacggcagttttgcttactggggccaagggactacagtgactgtttctagt

SEQ ID NO: 14 - DNA, 87 bp; single-stranded, linear
gagattgtgctaactcagtctccagccaccctgtctctcagcccaggagaaagggcgactctttcctgccaggccagccaaagtatt

SEQ ID NO: 15 - DNA, 75 bp; single-stranded, linear
ttagatgagaagccttggggcttgaccaggcctttgttgataccagtgtaggtggttgctaatactttggctggc

SEQ ID NO: 16 - DNA, 84 bp; single-stranded, linear
ccaaggcttctcatctaatatcgttcccagtccatctctgggatccccgccaggttcagtggcagtggatcagggacagatttc

SEQ ID NO: 17 - DNA, 81 bp; single-stranded, linear
gctgccactctgttgacagtaatagactgcaaaatcttcaggctccagactggagatagtgagggtgaaatctgtccctga

SEQ ID NO: 18 - DNA, 57 bp; single-stranded, linear
caacagagtggcagctggcctcacacgttcggaggggggaccaaggtggaaattaag

SEQ ID NO: 19 - DNA, 36 bp; single-stranded, linear
gcccaaccagccatggccgatattgtgctaactcag

SEQ ID NO: 20 - DNA, 20 bp; single-stranded, linear
acagttggtgcagcatcagc

SEQ ID NO: 21 - DNA, 36 bp; single-stranded, linear
acccctgtggcaaaagccgaagtgcagctggtggag

SEQ ID NO: 22 - DNA, 20 bp; single-stranded, linear
gatgggggtgtcgttttggc

SEQ ID NO: 23 - protein, 10 aa
YPYDVPDYAS

SEQ ID NO: 24 - DNA, 23 bp; single-stranded, linear
  misc_feature 11..13: /note= “”NNN“ represents a codon specifying any amino acid other than Lys.”
gggaacgatannngatgagaagc

(19) United States
(12) Patent Application Publication
(10) Pub. No.: US 2005/0069554 A1
(43) Pub. Date: Mar. 31, 2005

(54) Anti-alphavbeta3 recombinant human antibodies, nucleic acids encoding same and methods of use

(75) Inventor: William D. Huse, Del Mar, CA (US)

Correspondence Address: McDermott Will & Emery LLP;Suite 700, 4370 La Jolla Village Drive, San Diego, CA, 92122, US

(73) Assignee: Applied Molecular Evolution, Inc.

(21) Appl. No.: 10/959,871

(22) Filed: Oct. 5, 2004

Related U.S. Application Data
(63) Continuation of application No. 08/790,540, filed on Jan. 30, 1997, now abandoned.

(51) Int. Cl. A61K 39/395; C07H 21/04; C07K 16/46; C12P 21/08

(52) U.S. Cl. 424/178.1; 530/391.1; 536/23.53; 435/69.1; 435/320.1; 435/328

(57) ABSTRACT

The invention provides a LM609 grafted antibody exhibiting selective binding affinity to αᵥβ₃. The LM609 grafted antibody consists of at least one LM609 CDR grafted heavy chain polypeptide and at least one LM609 CDR grafted light chain polypeptide or functional fragment thereof. Nucleic acids encoding LM609 grafted heavy and light chains as well as nucleic acids encoding the parental non-human antibody LM609 are additionally provided. Functional fragments of such encoding nucleic acids are similarly provided. The invention also provides a method of inhibiting a function of αᵥβ₃. The method consists of contacting αᵥβ₃ with a LM609 grafted antibody or functional fragment thereof under conditions which allow binding to αᵥβ₃. Finally, the invention provides for a method of treating an αᵥβ₃-mediated disease. The method consists of administering an effective amount of a LM609 grafted antibody or functional fragment thereof under conditions which allow binding to αᵥβ₃.

### BACKGROUND OF THE INVENTION

The present invention relates generally to integrin mediated diseases and, more particularly, to nucleic acids encoding αᵥβ₃-inhibitory monoclonal antibodies and to CDR grafted αᵥβ₃-inhibitory antibodies for the therapeutic treatment of αᵥβ₃-mediated diseases.

Integrins are a class of cell adhesion receptors that mediate both cell-cell and cell-extracellular matrix adhesion events. Integrins consist of heterodimeric polypeptides where a single α chain polypeptide noncovalently associates with a single β chain. There are now about 14 distinct α chain polypeptides and at least about 8 different β chain polypeptides which constitute the integrin family of cell adhesion receptors. In general, different binding specificities and tissue distributions are derived from unique combinations of the α and β chain polypeptides or integrin subunits. The family to which a particular integrin is associated with is usually characterized by the β subunit. However, the ligand binding activity of the integrin is largely influenced by the α subunit. For example, vitronectin binding integrins contain the αᵥ integrin subunit.

It is now known that the vitronectin binding integrins consist of at least three different αᵥ containing integrins. These αᵥ containing integrins include αᵥβ₃, αᵥβ₁ and αᵥβ₅, all of which exhibit different ligand binding specificities. For example, in addition to vitronectin, αᵥβ₃ binds to a large variety of extracellular matrix proteins including fibronectin, fibrinogen, laminin, thrombospondin, von Willebrand factor, collagen, osteospontin and bone sialoprotein I. The integrin αᵥβ₁ binds to fibronectin, osteopontin and vitronectin whereas αᵥβ₅ is known to bind to vitronectin and osteopontin.

As cell adhesion receptors, integrins are involved in a variety of physiological processes including, for example, cell attachment, cell migration and cell proliferation. Different integrins play different roles in each of these biological processes and the inappropriate regulation of their function or activity can lead to various pathological conditions. For example, inappropriate endothelial cell proliferation during neovascularization of a tumor has been found to be mediated by cells expressing vitronectin binding integrins. In this regard, the inhibition of the vitronectin-binding integrin αᵥβ₃ also inhibits this process of tumor neovascularization. By this same criteria, αᵥβ₃ has also been shown to mediate the abnormal cell proliferation associated with restenosis and granulation tissue development in cutaneous wounds, for example. Additional diseases or pathological states mediated or influenced by αᵥβ₃ include, for example, metastasis, osteoporosis, age-related macular degeneration and diabetic retinopathy, and inflammatory diseases such as rheumatoid arthritis and psoriasis. Thus, agents which can specifically inhibit vitronectin-binding integrins would be valuable for the therapeutic treatment of diseases.

Many integrins mediate their cell adhesive functions by recognizing the tripeptide sequence Arg-Gly-Asp (RGD) found within a large number of extracellular matrix proteins. A variety of approaches have attempted to model agents after this sequence to target a particular integrin-mediated pathology. Such approaches include, for example, the use of RGD-containing peptides and peptide analogues which rely on specificity to be conferred by the sequences flanking the RGD core tripeptide sequence. Although there has been some limited success, most RGD-based inhibitors have been shown to be, at most, selective for the targeted integrin and therefore exhibit some cross-reactivity to other non-targeted integrins. Such cross-reactive inhibitors therefore lack the specificity required for use as an efficacious therapeutic. This is particularly true for previously identified inhibitors of the integrin αᵥβ₃.

Monoclonal antibodies on the other hand exhibit the specificity required to be used as an effective therapeutic. Antibodies also have the advantage in that they can be routinely generated against essentially any desired antigen. Moreover, with the development of combinatorial libraries, antibodies can now be produced faster and more efficiently than by previously used methods within the art. The use of combinatorial methodology also allows for the selection of the desired antibody along with the simultaneous isolation of the encoding heavy and light chain nucleic acids. Thus, further modification can be performed to the combinatorial antibody without the incorporation of an additional cloning step.

Regardless of the potential advantages associated with the use of monoclonal antibodies as therapeutics, these molecules nevertheless have the drawback in that they are almost exclusively derived from non-human mammalian organisms. Therefore, their use as therapeutics is limited by the fact that they will normally elicit a host immune response. Methods for substituting the antigen binding site or complementarity determining regions (CDRs) of the non-human antibody into a human framework have been described. Such methods vary in terms of which amino acid residues should be substituted as the CDR as well as which framework residues should be changed to maintain binding specificity. In this regard, it is understood that proper orientation of the β sheet architecture, correct packing of the heavy and light chain interface and appropriate conformation of the CDRs are all important for preserving antigen specificity and affinity within the grafted antibody. However, all of these methods require knowledge of the nucleotide and amino acid sequence of the non-human antibody and the availability of an appropriately modeled human framework.

Thus, there exists a need for the availability of nucleic acids encoding integrin inhibitory antibodies which can be used as compatible therapeutics in humans. For αᵥβ₃-mediated diseases, the present invention satisfies this need and provides related advantages as well.

### SUMMARY OF THE INVENTION

The invention provides a LM609 grafted antibody exhibiting selective binding affinity to αᵥβ₃. The LM609 grafted antibody consists of at least one LM609 CDR grafted heavy chain polypeptide and at least one LM609 CDR grafted light chain polypeptide or functional fragment thereof. Nucleic acids encoding LM609 grafted heavy and light chains as well as nucleic acids encoding the parental non-human antibody LM609 are additionally provided. Functional fragments of such encoding nucleic acids are similarly provided. The invention also provides a method of inhibiting a function of αᵥβ₃. The method consists of contacting αᵥβ₃ with a LM609 grafted antibody or functional fragment thereof under conditions which allow binding to αᵥβ₃. Finally, the invention provides for a method of treating an αᵥβ₃-mediated disease. The method consists of administering an effective amount of a LM609 grafted antibody or functional fragment thereof under conditions which allow binding to αᵥβ₃.

### BRIEF DESCRIPTION OF THE DRAWINGS

FIG. 1 shows the nucleotide and deduced amino acid sequence of the variable region of the LM609 grafted antibody. FIG. 1A shows the nucleotide and deduced amino acid sequences for the LM609 grafted heavy chain variable region (Gln1-Ser117; SEQ ID NOS:1 and 2, respectively) while FIG. 1B shows the nucleotide and deduced amino acid sequences for the LM609 grafted light chain variable region (Glu1-Lys107; SEQ ID NOS:3 and 4, respectively).

FIG. 2 shows the nucleotide and deduced amino acid sequence of the variable region of the monoclonal antibody LM609. FIG. 2A shows the nucleotide and deduced amino acid sequence of the LM609 heavy chain varible region (SEQ ID NOS:5 and 6, respectively). The variable region extends from amino acid Glu1 to Ala117. FIG. 2B shows the nucleotide and deduced amino acid sequence of the LM609 light chain variable region (SEQ ID NOS:7 and 8, respectively). The variable region of the light chain extends from amino acid Asp1 to Lys107.

FIG. 3 shows the competitive inhibition of LM609 IgG binding to the integrin αᵥβ₃ with recombinant LM609 Fab. Soluble recombinant murine LM609 Fab fragments were prepared from periplasmic fractions of M13 bacteriophage clones muLM609M13 12 and muLM609M13 29. The periplasm samples were serially diluted, mixed with either 1 ng/ml, 5 ng/ml, or 50 ng/ml of LM609 IgG and then incubated in 96 well plates coated with purified αᵥβ₃. Plates were washed and bound LM609 IgG detected with goat anti-murine Fc specific antibody conjugated to alkaline phosphatase. Fab produced by clone muLM609M13 12 inhibits both 1 ng/ml and 5 ng/ml LM609 IgG binding at all concentrations of Fab greater than 1:27 dilution.

### DETAILED DESCRIPTION OF THE INVENTION

The invention is directed to nucleic acids encoding the monoclonal antibody (MAb) LM609. This antibody specifically recognizes the integrin αᵥβ₃ and inhibits its functional activity. The invention is also directed to nucleic acids encoding and to polypeptides comprising non-murine forms of LM609 termed LM609 grafted antibodies. A LM609 grafted antibody retains the binding specificity and inhibitory activity of its parent murine antibody LM609.

In one embodiment, the hybridoma expressing LM609 was used as a source to generate and clone cDNAs encoding LM609. The heavy and light chain encoding cDNAs were sequenced and their CDR regions as defined by Kabat et al., supra were substituted into a human antibody framework to generate the non-murine form of the antibody. As an antibody having CDRs grafted to a human acceptor framework, it is unlikely that LM609 grafted antibodies will elicit a host immune response and can therefore be advantageously used for the treatment of αᵥβ₃-mediated diseases.

As used herein, the term “monoclonal antibody LM609” or “LM609” is intended to mean the murine monoclonal antibody specific for the integrin αᵥβ₃ which is described by Cheresh, D. A. Proc. Natl. Acad. Sci. USA 84:6471-6475 (1987) and by Cheresh and Spiro J. Biol. Chem. 262:17703-17711 (1987). LM609 was produced against and is reactive with the M21 cell adhesion receptor now known as the integrin αᵥβ₃ LM609 inhibits the attachment of M21 cells to αᵥβ₃ ligands such as vitronectin, fibrinogen and von Willebrand factor (Cheresh and Spiro, supra) and is also an inhibitor of αᵥβ₃-mediated pathologies such as tumor induced angiogenesis (Brooks et al. Cell 79:1157-1164 (1994), granulation tissue development in cutaneous wound (Clark et al., Am. J. Pathology, 148:1407-1421 (1996)) and smooth muscle cell migration such as that occurring during restenosis (Choi et al., J. Vascular Surg., 19:125-134 (1994); Jones et al., Proc. Natl. Acad. Sci. 93:2482-2487 (1996)).

As used herein, the term “LM609 grafted antibody” is intended to refer to a non-mouse antibody or functional fragment thereof having substantially the same heavy and light chain CDR amino acid sequences as found in LM609 and absent of the substitution of LM609 amino acid residues outside of the CDRs as defined by Kabat et al., U.S. Dept. of Health and Human Services, “Sequences of Proteins of Immunological Interest” (1983). The term “LM609 grafted antibody” or “LM609 grafted” when used in reference to heavy or light chain polypeptides is intended to refer to a non-mouse heavy or light chain or functional fragment thereof having substantially the same heavy or light chain CDR amino acid sequences as found in the heavy or light chain of LM609, respectively, and also absent of the substitution of LM609 residues outside of the CDRs as defined by Kabat et al., supra. When used in reference to a functional fragment, not all LM609 CDRs need to be represented. Rather, only those CDRs that would normally be present in the antibody portion that corresponds to the functional fragment are intended to be referenced as the LM609 CDR amino acid sequences in the LM609 grafted functional fragment. Similarly, the term “LM609 grafted antibody” or “LM609 grafted” used in reference to an encoding nucleic acid is intended to refer to a nucleic acid encoding a non-mouse antibody or functional fragment being absent of the substitution of LM609 amino acids outside of the CDRs as defined by Kabat et al., supra and having substantially the same nucleotide sequence as the heavy and light chain CDR nucleotide sequences and encoding substantially the same CDR amino acid sequences as found in LM609 and as defined by Kabat et al., supra.

The term “grafted antibody” or “grafted” when used in reference to heavy or light chain polypeptides or functional fragments thereof is intended to refer to a heavy or light chain or functional fragment thereof having substantially the same heavy or light chain of a donor antibody, respectively, and also absent of the substitution of donor amino acid residues outside of the CDRs as defined by Kabat et al., supra. When used in reference to a functional fragment, not all donor CDRs need to be represented. Rather, only those CDRs that would normally be present in the antibody portion that corresponds to the functional fragment are intended to be referenced as the donor CDR amino acid sequences in the functional fragment. Similarly, the term “grafted antibody” or “grafted” when used in reference to an encoding nucleic acid is intended to refer to a nucleic acid encoding an antibody or functional fragment, being absent of the substitution of donor amino acids outside of the CDRs as defined by Kabat et al., supra and having substantially the same nucleotide sequence as the heavy and light chain CDR nucleotide sequences and encoding substantially the same CDR amino acid sequences as found in the donor antibody and as defined by Kabat et al., supra.

The meaning of the above terms are intended to include minor variations and modifications of the antibody so long as its function remains uncompromised. Functional fragments such as Fab, F(ab) 2, Fv, single chain Fv (scFv) and the like are similarly included within the definition of the terms LM609 and LM609 grafted antibody. Such functional fragments are well known to those skilled in the art. Accordingly, the use of these terms in describing functional fragments of LM609 or LM609 grafted antibodies are intended to correspond to the definitions well known to those skilled in the art. Such terms are described in, for example, Harlow and Lane, Antibodies: A Laboratory Manual, Cold Spring Harbor Laboratory, New York (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, Second Ed., Wiley-Liss, Inc., New York, N.Y. (1990).

As with the above terms used for describing functional fragments of LM609 and a LM609 grafted antibody, the use of terms which reference other LM609, or LM609 grafted antibody domains, functional fragments, regions, nucleotide and amino acid sequences and polypeptides or peptides, is similarly intended to fall within the scope of the meaning of each term as it is known and used within the art. Such terms include, for example, “heavy chain polypeptide” or “heavy chain”, “light chain polypeptide” or “light chain”, “heavy chain variable region” (V_{H}) and “light chain variable region” (V_{L}) as well as the term “complementarity determining region” (CDR).

In the case where there are two or more definitions of a term which is used and/or accepted within the art, the definition of the term as used herein is intended to include all such meanings unless explicitly stated to the contrary. A specific example is the use of the term “CDR” to describe the non-contiguous antigen combining sites found within the variable region of both heavy and light chain polypeptides. This particular region has been described by Kabat et al., supra, and by Chothia et al., J. Mol. Biol. 196:901-917 (1987) and by MacCallum et al., J. Mol. Biol. 262:732-745 (1996) where the definitions include overlapping or subsets of amino acid residues when compared against each other. Nevertheless, application of either definition to refer to a CDR of LM609, LM609 grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Clothia et al., supra
³Residue numbering follows the nomenclature of MacCallum et al., supra

As used herein, the term “substantially” or “substantially the same” when used in reference to a nucleotide or amino acid sequence is intended to mean that the nucleotide or amino acid sequence shows a considerable degree, amount or extent of sequence identity when compared to a reference sequence. Such considerable degree, amount or extent of sequence identity is further considered to be significant and meaningful and therefore exhibit characteristics which are definitively recognizable or known. Thus, a nucleotide sequence which is substantially the same nucleotide sequence as a heavy or light chain of LM609, or a LM609 grafted antibody including fragments thereof, refers to a sequence which exhibits characteristics that are definitively known or recognizable as encoding or as being the amino acid sequence of LM609 or a LM609 grafted antibody. Minor modifications thereof are included so long as they are recognizable as a LM609 or a LM609 grafted antibody sequence. Similarly, an amino acid sequence which is substantially the same amino acid sequence as a heavy or light chain of LM609 grafted antibody or functional fragment thereof, refers to a sequence which exhibits characteristics that are definitively known or recognizable as representing the amino acid sequence of a LM609 grafted antibody and minor modifications thereof.

As used herein, the term “fragment” when used in reference to a nucleic acid encoding LM609 or a LM609 grafted antibody is intended to mean a nucleic acid having substantially the same sequence as a portion of a nucleic acid encoding LM609 or a LM609 grafted antibody. The nucleic acid fragment is sufficient in length and sequence to selectively hybridize to a LM609 or a LM609 grafted antibody encoding nucleic acid or a nucleotide sequence that is complementary to an LM609 or LM609 grafted antibody encoding nucleic acid. Therefore, fragment is intended to include primers for sequencing and polymerase chain reaction (PCR) as well as probes for nucleic acid blot or solution hybridization. The meaning of the term is also intended to include regions of nucleotide sequences that do not directly encode LM609 polypeptides such as the introns, and the untranslated region sequences of the LM609 encoding gene.

As used herein, the term “functional fragment” when used in reference to a LM609 grafted antibody or to heavy or light chain polypeptides thereof is intended to refer to a portion of a LM609 grafted antibody including heavy or light chain polypeptides which still retains some or all or the αᵥβ₃ binding activity, αᵥβ₃ binding specificity and/or integrin αᵥβ₃-inhibitory activity. Such functional fragments can include, for example, antibody functional fragments such as Fab, F(ab)₂, Fv, single chain Fv (scFv). Other functional fragments can include, for example, heavy or light chain polypeptides, variable region polypeptides or CDR polypeptides or portions thereof so long as such functional fragments retain binding activity, specificity or inhibitory activity. The term is also intended to include polypeptides encompassing, for example, modified forms of naturally occurring amino acids such as D-stereoisomers, non-naturally occurring amino acids, amino acid analogues and mimetics so long as such polypeptides retain functional activity as defined above.

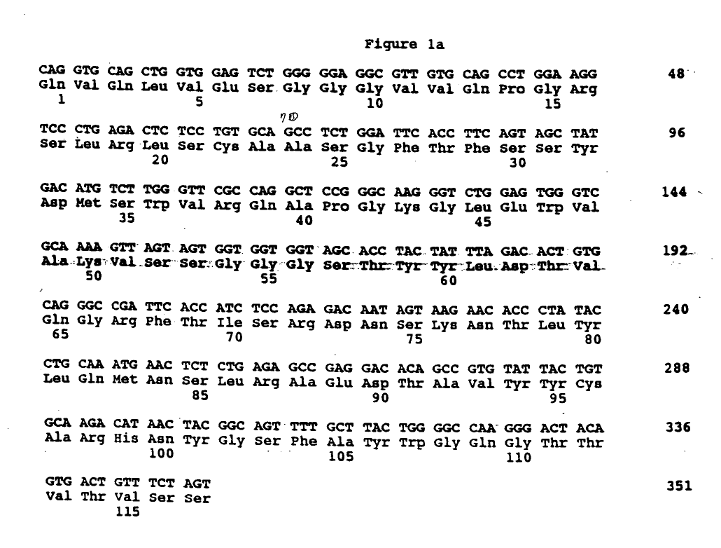

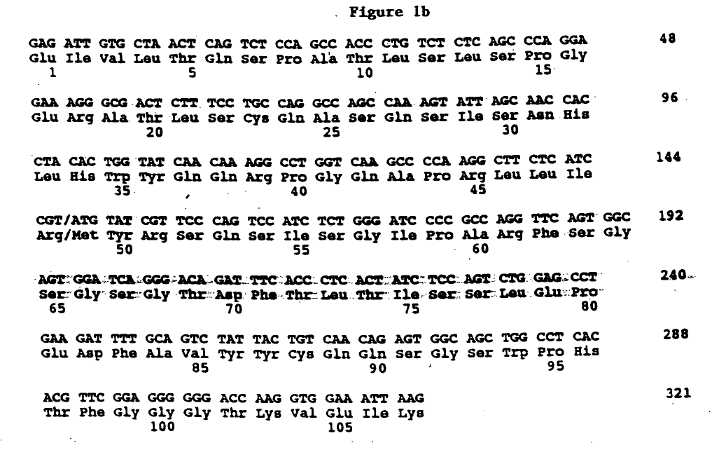

The invention provides a nucleic acid encoding a heavy chain polypeptide for a LM609 grafted antibody or a functional fragment thereof. Also provided is a nucleic acid encoding a light chain polypeptide for a LM609 grafted antibody or a functional fragment thereof. The nucleic acids consist of substantially the same heavy or light chain variable region nucleotide sequences as those shown in FIGS. 1A and 1B (SEQ ID NOS:1 and 3, respectively) or a fragment thereof.

A LM609 grafted antibody, including functional fragments thereof, is a non-mouse antibody which exhibits substantially the same binding activity, binding specificity and inhibitory activity as LM609. The LM609 grafted antibody Fv fragments described herein are produced by functionally replacing CDRs as defined by Kabat et al., hereinafter referred to as “Kabat CDRs,” within human heavy and light chain variable region polypeptides with the Kabat CDRs derived from LM609. Functional replacement of the CDRs was performed by recombinant methods known to those skilled in the art. Such methods are commonly referred to as CDR grafting and are the subject matter of U.S. Pat. No. 5,225,539. Such methods can also be found described in “Protein Engineering of Antibody Molecules for Prophylactic and Therapeutic Applications in Man,” Clark, M. (ed.), Nottingham, England: Academic Titles (1993).

Substitution of amino acid residues outside of the Kabat CDRs can additionally be performed to maintain or augment beneficial binding properties of LM609 grafted antibodies so long as such amino acid substitutions do not correspond to a donor amino acid at that particular position. Such substitutions allow for the modulation of binding properties without imparting any mouse sequence characteristics onto the antibody outside of the Kabat CDRs. Although the production of such antibodies is described herein with reference to LM609 grafted antibodies, the substitution of such non-donor amino acids outside of the Kabat CDRs can be utilized for the production of essentially any grafted antibody. The production of LM609 grafted antibodies is described further below in Example II.

Briefly, LM609 nucleic acid fragments having substantially the same nucleotide and encoding substantially the same amino acid sequence of each of the heavy and light chain CDRs were synthesized and substituted into each of the respective human chain encoding nucleic acids. Modifications were performed within the non-Kabat CDR framework region. These individual changes were made by generating a population of Kabat CDR grafted heavy and light chain variable regions wherein all possible non-donor amino acid changes outside of the Kabat CDRs were represented and then selecting the appropriate antibody by screening the population for binding activity. This screen resulted in the selection of the LM609 grafted antibodies described herein.

The nucleotide sequences of the LM609 grafted heavy and light chain variable regions are shown in FIGS. 1A and 1B, respectively. These sequences correspond substantially to those that encode the heavy and light chain variable region polypeptides of a LM609 grafted antibody. These nucleic acids are intended to include both the sense and anti-sense strands of the LM609 grafted antibody encoding sequences. Single- and double-stranded nucleic acids are similarly included as well as non-coding portions of the nucleic acid such as introns, 5′- and 3′-untranslated regions and regulatory sequences of the gene for example.

As shown in FIG. 1A, the LM609 grafted heavy chain variable region polypeptide is encoded by a nucleic acid of about 351 nucleotides in length which begins at the amino terminal Gln1 residue of the variable region through to Ser117. This heavy chain variable region encoding nucleic acid is joined to a human IgG1 constant region to yield a coding region of 1431 nucleotides which encodes a heavy chain polypeptide of 477 total amino acids. Shown in FIG. 1B is the LM609 grafted light chain variable region polypeptide which is encoded by a nucleic acid of about 321 nucleotides in length beginning at the amino terminal Glu1 residue of the variable region through to Lys107. This light chain variable region nucleic acid is joined to a human kappa construct region to yield a coding region of 642 nucleotides which code for a light chain polypeptide of 214 total amino acids.

Minor modification of these nucleotide sequences are intended to be included as LM609 grafted heavy and light chain encoding nucleic acids and their functional fragments. Such minor modifications include, for example, those which do not change the encoded amino acid sequence due to the degeneracy of the genetic code as well as those which result in only a conservative substitution of the encoded amino acid sequence. Conservative substitutions of encoded amino acids include, for example, amino acids which belong within the following groups: (1) non-polar amino acids (Gly, Ala, Val, Leu, and Ile); (2) polar neutral amino acids (Cys, Met, Ser, Thr, Asn, and Gln); (3) polar acidic amino acids (Asp and Glu); (4) polar basic amino acids (Lys, Arg and His); and (5) aromatic amino acids (Phe, Trp, Tyr, and His). Other minor modifications are included within the nucleic acids encoding LM609 grafted heavy and light chain polypeptides so long as the nucleic acid or encoded polypeptides retain some or all of their function as described herein.

Thus, the invention also provides a nucleic acid encoding a LM609 grafted heavy chain or functional fragment thereof wherein the nucleic acid encodes substantially the same LM609 grafted heavy chain variable region amino acid sequence as that shown in FIG. 1A (SEQ ID NO:2) or a fragment thereof. Similarly, the invention also provides a nucleic acid encoding a LM609 grafted light chain or functional fragment thereof wherein the nucleic acid encodes substantially the same light chain variable region amino acid sequence as that shown in FIG. 1B (SEQ ID NO:4) or a fragment thereof.

In addition to conservative substitutions of amino acids, minor modifications of the LM609 grafted antibody encoding nucleotide sequences which allow for the functional replacement of amino acids are also intended to be included within the definition of the term. The substitution of functionally equivalent amino acids encoded by the LM609 grafted antibody nucleotide sequences is routine and can be accomplished by methods known to those skilled in the art. Briefly, the substitution of functionally equivalent amino acids can be made by identifying the amino acids which are desired to be changed, incorporating the changes into the encoding nucleic acid and then determining the function of the recombinantly expressed and modified LM609 grafted polypeptide or polypeptides. Rapid methods for making and screening multiple simultaneous changes are well known within the art and can be used to produce a library of encoding nucleic acids which contain all possible or all desired changes and then expressing and screening the library for LM609 grafted polypeptides which retain function. Such methods include, for example, codon based mutagenesis, random oligonucleotide synthesis and partially degenerate oligonucleotide synthesis.

Codon based mutagenesis is the subject matter of U.S. Pat. Nos. 5,264,563 and 5,523,388 and is advantageous for the above procedures since it allows for the production of essentially any and all desired frequencies of encoded amino acid residues at any and all particular codon positions within an oligonucleotide. Such desired frequencies include, for example, the truly random incorporation of all twenty amino acids or a specified subset thereof as well as the incorporation of a predetermined bias of one or more particular amino acids so as to incorporate a higher or lower frequency of the biased residues compared to other incorporated amino acid residues. Random oligonucleotide synthesis and partially degenerate oligonucleotide synthesis can similarly be used for producing and screening for functionally equivalent amino acid changes. However, due to the degeneracy of the genetic code, such methods will incorporate redundancies at a desired amino acid position. Random oligonucleotide synthesis is the coupling of all four nucleotides at each nucleotide position within a codon whereas partially degenerate oligonucleotide synthesis is the coupling of equal portions of all four nucleotides at the first two nucleotide positions, for example, and equal portions of two nucleotides at the third position. Both of these latter synthesis methods can be found described in, for example, Cwirla et al., Proc. Natl. Acad. Sci. USA 87:6378-6382, (1990) and Devlin et al., Science 249:404-406, (1990).

Identification of amino acids to be changed can be accomplished by those skilled in the art using current information available regarding the structure and function of antibodies as well as available and current information encompassing methods for CDR grafting procedures.

Using the above described methods known within the art, any or all of the non-identical amino acids can be changed either alone or in combination with amino acids at different positions to incorporate the desired number of amino acid substitutions at each of the desired positions. The LM609 grafted polypeptides containing the desired substituted amino acids are then produced and screened for retention or augmentation of function compared to the unsubstituted LM609 grafted polypeptides. Production of the substituted LM609 grafted polypeptides can be accomplished by, for example, recombinant expression using methods known to those skilled in the art. Those LM609 grafted polypeptides which exhibit retention or augmentation of function compared to unsubstituted LM609 grafted polypeptides are considered to contain minor modifications of the encoding nucleotide sequence which result in the functional replacement of one or more amino acids.

The functional replacement of amino acids is beneficial when producing grafted antibodies having human framework sequences since it allows for the rapid identification of equivalent amino acid residues without the need for structural information or the laborious procedures necessary to assess and identify which amino acid residues should be considered for substitution in order to successfully transfer binding function from the donor. Moreover, it eliminates the actual step-wise procedures to change and test the amino acids identified for substitution. Essentially, using the functional replacement approach described above, all non-identical amino acid residues between the donor and the human framework can be identified and substituted with any or all other possible amino acid residues, excluding the corresponding donor amino acid, at each non-identical position to produce a population of substituted polypeptides containing all possible or all desired permutations and combinations. The population of substituted polypeptides can then be screened for those substituted polypeptides which retain function. Using the codon based mutagenesis procedures described above, the generation of a library of substituted amino acid residues and the screening of functionally replaced residues has been used for the rapid production of grafted therapeutic antibodies as well as for the rapid alteration of antibody affinity. Such procedures are exemplified in, for example, Rosok et al., J. Biol. Chem. 271:22611-22618 (1996) and in Glaser et al., J. Immunol. 149:3903-3913 (1992), respectively.

The invention further provides fragments of LM609 grafted heavy and light chain encoding nucleic acids wherein such fragments consist substantially of the same nucleotide or amino acid sequence as the LM609 grafted variable region of the heavy or light chain polypeptides. The variable region of the heavy chain polypeptide consists essentially of nucleotides 1-351 and of amino acid residues Gln1 to Ser117 of FIG. 1A (SEQ ID NOS:1 and 2, respectively). The variable region of the light chain polypeptide consists essentially of nucleotides 1-321 and of amino acid residues Glu1 to Lys107 of FIG. 1B (SEQ ID NOS:3 and 4, respectively). The termini of such variable region encoding nucleic acids is not critical so long as the intended purpose and function remains the same.

Fragments additional to the variable region nucleic acid fragments are provided as well. Such fragments include, for example, nucleic acids consisting substantially of the same nucleotide sequence as a CDR of a LM609 grafted heavy or light chain polypeptide. Sequences corresponding to the LM609 grafted CDRs include, for example, those regions defined by Kabat et al., supra, and/or those regions defined by Chothia et al., supra, as well as those defined by MacCallum et al., supra. The LM609 grafted CDR fragments for each of the above definitions correspond to the nucleotides set forth below in Table 2. The nucleotide sequence numbering is taken from the primary sequence shown in FIGS. 1A and 1B (SEQ ID NOS:1 and 3) and conforms to the definitions previously set forth in Table 1.

TABLE 2
LM609 Grafted CDR Nucleotide Residues

| | Kabat | Chothia | MacCallum |
|---|---|---|---|
| V_{H} CDR1 | 91-105 | 76-96 | 88-105 |
| V_{H} CDR2 | 148-198 | 157-168 | 139-177 |
| V_{H} CDR3 | 295-318 | 298-315 | 289-315 |
| V_{L} CDR1 | 70-102 | 76-96 | 88-108 |
| V_{L} CDR2 | 148-168 | 148-156 | 136-165 |
| V_{L} CDR3 | 265-291 | 271-288 | 265-288 |

Similarly, the LM609 grafted CDR fragments for each of the above definitions correspond to the amino acid residues set forth below in Table 3. The amino acid residue number is taken from the primary sequence shown in FIGS. 1A and 1B (SEQ ID NOS:2 and 4) and conforms to the definitions previously set forth in Table 1.

TABLE 3
LM609 Grafted CDR Amino Acid Residues

| | Kabat | Chothia | MacCallum |
|---|---|---|---|
| V_{H} CDR1 | Ser31-Ser35 | Gly26-Tyr32 | Ser30-Ser35 |
| V_{H} CDR2 | Lys50-Gly66 | Ser53-Gly56 | Trp47-Tyr59 |
| V_{H} CDR3 | His99-Tyr106 | Asn100-Ala105 | Ala97-Ala105 |
| V_{L} CDR1 | Gln24-His34 | Ser26-His32 | Ser30-Tyr36 |
| V_{L} CDR2 | Tyr50-Ser56 | Tyr50-Ser52 | Leu46-Ile55 |
| V_{L} CDR3 | Gln89-Thr97 | Ser91-His96 | Gln89-His96 |

Thus, the invention also provides nucleic acid fragments encoding substantially the same amino acid sequence as a CDR of a LM609 grafted heavy or light chain polypeptide.

Nucleic acids encoding LM609 grafted heavy and light chain polypeptides and fragments thereof are useful for a variety of diagnostic and therapeutic purposes. For example, the LM609 grafted nucleic acids can be used to produce LM609 grafted antibodies and functional fragments thereof having binding specificity and inhibitory activity against the integrin αᵥβ₃. The antibody and functional fragments thereof can be used for the diagnosis or therapeutic treatment of αᵥβ₃-mediated disease. A LM609 grafted antibody and functional fragments thereof can be used, for example, to inhibit binding activity or other functional activities of α₁β₃ that are necessary for progression of an αᵥβ₃-mediated disease. Other functional activities necessary for progression of αᵥβ₃-mediated disease include, for example, the activation of αᵥβ₃, αᵥβ₃-mediated signal transduction and the αᵥβ₃-mediated prevention of apoptosis. Advantageously, however, a LM609 grafted antibody comprises non-mouse framework amino acid sequences and as such is less antigenic in regard to the induction of a host immune response. The LM609 grafted antibody nucleic acids of the invention can also be used to model functional equivalents of the encoded heavy and light chain polypeptides.

Thus, the invention provides LM609 grafted heavy chain and LM609 grafted light chain polypeptides or functional fragments thereof. The LM609 grafted heavy chain polypeptide exhibits substantially the same amino acid sequence as that shown in FIG. 1A (SEQ ID NO:2) or functional fragment thereof whereas the LM609 grafted light chain polypeptide exhibits substantially the same amino acid sequence as that shown in FIG. 1B (SEQ ID NO:4) or functional fragment thereof. Also provided is a LM609 grafted antibody or functional fragment thereof. The antibody is generated from the above heavy and light chain polypeptides or functional fragments thereof and exhibits selective binding affinity to αᵥβ₃.

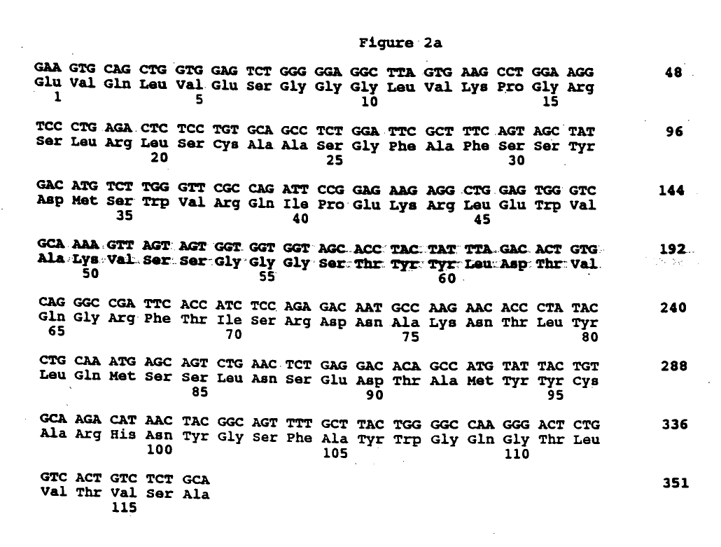

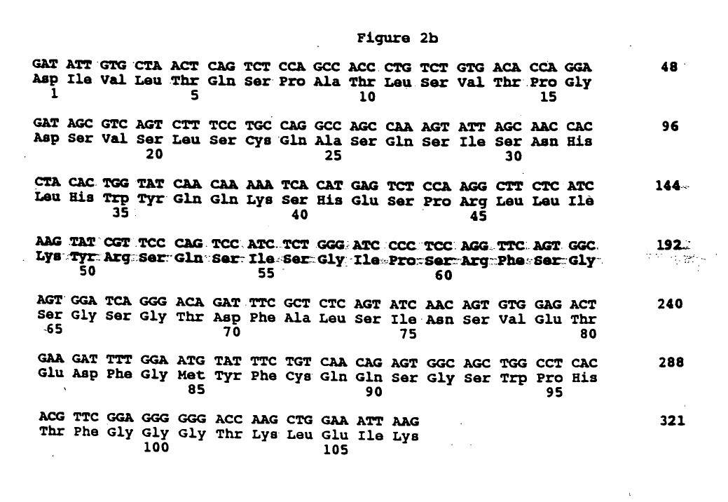

The invention provides a nucleic acid encoding a heavy chain polypeptide for monoclonal antibody LM609 or functional fragment thereof. Also provided is a nucleic acid encoding a light chain polypeptide for monoclonal antibody LM609 or a functional fragment thereof. The nucleic acids consist of substantially the same heavy or light chain variable region nucleotide sequences as that shown in FIGS. 2A and 2B (SEQ ID NOS:5 and 7, respectively) or a fragment thereof.

As described previously, monoclonal antibody LM609 has been shown in the art to have binding activity to the integrin αᵥβ₃. Although specificity can in principle be generated towards essentially any target, LM609 is an integrin inhibitory antibody that exhibits substantial specificity and inhibitory activity to a single member within an integrin family. In this case, LM609 exhibits substantial specificity and inhibitory activity to the αᵥβ₃ integrin within the β₃ family. The amino acid or nucleotide sequence of monoclonal antibody LM609 has never been previously isolated and characterized.

The isolation and characterization of LM609 encoding nucleic acids was performed by techniques known to those skilled in the art and which are described further below in the Examples. Briefly, cDNA from hybridoma LM609 was generated and used as the source for which to isolate LM609 encoding nucleic acids. Isolation was performed by first determining the N-terminal amino acid sequence for each of the heavy and light chain polypeptides and then amplifying by PCR the antibody encoding sequences from the cDNA. The 5′ primers were reverse translated to correspond to the newly determined N-terminal amino acid sequences whereas the 3′ primers corresponded to sequences substantially similar to antibody constant region sequences. Amplification and cloning of the products resulted in the isolation of the nucleic acids encoding heavy and light chains of LM609.

The nucleotide sequences of the LM609 heavy and light chain variable region sequences are shown in FIGS. 2A and 2B, respectively. These sequences correspond substantially to those that encode the variable region heavy and light chain polypeptides of LM609. As with the LM609 grafted antibody nucleic acids, these LM609 nucleic acids are intended to include both sense and anti-sense strands of the LM609 encoding sequences. Single- and double-stranded nucleic acids are also included as well as non-coding portions of the nucleic acid such as introns, 5′- and 3′-untranslated regions and regulatory sequences of the gene for example.

As shown in FIG. 2A, the LM609 heavy chain variable region polypeptide is encoded by a nucleic acid of about 351 nucleotides in length which begins at the amino terminal Glu1 residue of the variable region through to Ala 117. The murine LM609 antibody heavy chain has an IgG2a constant region. Shown in FIG. 2B is the LM609 light chain variable region polypeptide which is encoded by a nucleic acid of about 321 nucleotides in length which begins at the amino terminal Aspl residue of the variable region through to Lys 107. In the functional antibody, LM609 has a kappa light chain constant region.

As with the LM609 grafted antibody nucleic acids, minor modifications of these LM609 nucleotide sequences are intended to be included as heavy and light chain LM609 encoding nucleic acids. Such minor modifications are included within the nucleic acids encoding LM609 heavy and light chain polypeptides so long as the nucleic acids or encoded polypeptides retain some or all of their function as described.

Thus, the invention also provides a nucleic acid encoding a LM609 heavy chain or functional fragment wherein the nucleic acid encodes substantially the same variable region amino acid sequence of monoclonal antibody LM609 as that shown in FIG. 2A (SEQ ID NO:6) or a fragment thereof. Similarly, the invention also provides a nucleic acid encoding a LM609 light chain or functional fragment wherein the nucleic acid encodes substantially the same variable region amino acid sequence of monoclonal antibody LM609 as that shown in FIG. 2B (SEQ ID NO:8) or a fragment thereof.

The invention further provides fragments of LM609 heavy and light chain encoding nucleic acids wherein such fragments consist substantially of the same nucleotide or amino acid sequence as the variable region of LM609 heavy or light chain polypeptides. The variable region of the LM609 heavy chain polypeptide consists essentially of nucleotides 1-351 and of amino acid residues Glu1 to Ala117 of FIG. 2A (SEQ ID NOS:5 and 6, respectively). The variable region of the LM609 light chain polypeptide consists essentially of nucleotides 1-321 and of amino acid residues Asp1 to lys107′ of FIG. 2B (SEQ ID NOS:7 and 8, respectively). The termini of such variable region encoding nucleic acids is not critical so long as the intended purpose and function remains the same. Such intended purposes and functions include, for example, use for the production of recombinant polypeptides or as hybridization probes for heavy and light chain variable region sequences.

Fragments additional to the variable region nucleic acid fragments are provided as well. Such fragments include, for example, nucleic acids consisting substantially of the same nucleotide sequence as a CDR of a LM609 heavy or light chain polypeptide. Sequences corresponding to the LM609 CDRs include, for example, those regions within the variable region which are defined by Kabat et al., supra, and/or those regions within the variable regions which are defined by Chothia et al., supra, as well as those regions defined by MacCallum et al., supra. The LM609 CDR fragments for each of the above definitions correspond to the nucleotides set forth below in Table 4. The nucleotide sequence numbering is taken from the primary sequence shown in FIGS. 2A and 2B (SEQ ID NOS:5 and 7) and conforms to the definitions previously set forth in Table 1.

TABLE 4
LM609 CDR Nucleotide Residues

| | Kabat | Chothia | MacCallum |
|---|---|---|---|
| V_{H} CDR1 | 91-105 | 76-96 | 88-105 |
| V_{H} CDR2 | 148-198 | 157-168 | 139-177 |
| V_{H} CDR3 | 295-318 | 298-315 | 289-315 |
| V_{L} CDR1 | 70-102 | 76-96 | 88-108 |
| V_{L} CDR2 | 148-168 | 148-156 | 136-165 |
| V_{L} CDR3 | 265-291 | 271-288 | 265-288 |

Similarly, the LM609 CDR fragments for each of the above definitions correspond to the amino acid residues set forth below in Table 5. The amino acid residue numbering-is taken from the primary sequence shown in FIGS. 2A and 2B (SEQ ID NOS:6 and 8) and conforms to the definitions set forth in Table 1.

TABLE 5
LM609 CDR Amino Acid Residues

| | Kabat | Chothia | MacCallum |
|---|---|---|---|
| V_{H} CDR1 | Ser31-Ser35 | Gly26-Tyr32 | Ser30-Ser35 |
| V_{H} CDR2 | Lys50-Gly66 | Ser53-Gly56 | Trp47-Tyr59 |
| V_{H} CDR3 | His99-Tyr106 | Asn100-Ala105 | Ala97-Ala105 |
| V_{L} CDR1 | Gln24-His34 | Ser26-His32 | Ser30-Tyr36 |
| V_{L} CDR2 | Tyr50-Ser56 | Tyr50-Ser52 | Leu46-Ile55 |
| V_{L} CDR3 | Gln89-Thr97 | Ser91-His96 | Gln89-His96 |

Nucleic acids encoding LM609 heavy and light chain polypeptides and fragments thereof are useful for a variety of diagnostic and therapeutic purposes. For example, the LM609 nucleic acids can be used to produce recombinant LM609 antibodies and functional fragments thereof having binding specificity and inhibitory activity against the integrin βᵥβ₃. The antibody and functional fragments thereof can be used to determine the presence or absence of αᵥβ₃ in a sample to diagnose the susceptibility or occurrence of an αᵥβ₃-mediated disease. Alternatively, the recombinant LM609 antibodies and functional fragments thereof can be used for the therapeutic treatment of αᵥβ₃ mediated diseases or pathological state. As with a LM609 grafted antibody, recombinant LM609 and functional fragments thereof can be used to inhibit the binding activity or other functional activities of αᵥβ₃ that are necessary for progression of the αᵥβ₃-mediated disease or pathological state.

The LM609 nucleic acids of the invention can also be used to model functional equivalents of the encoded heavy and light chain polypeptides. Such functional equivalents can include, for example, synthetic analogues or mimics of the encoded polypeptides or functional fragments thereof. A specific example would include peptide mimetics of the LM609 CDRs that retain some or substantially the same binding or inhibitory activity of LM609. Additionally, the LM609 encoding nucleic acids can be used to engineer and produce nucleic acids which encode modified forms or derivatives of the antibody LM609, its heavy and light chain polypeptides and functional fragments thereof. As described previously, such modified forms or derivatives include, for example, non-mouse antibodies, their corresponding heavy and light chain polypeptides and functional fragments thereof which exhibit substantially the same binding and inhibitory activity as LM609.

The invention also provides a method of treating an αᵥβ₃-mediated disease. The method consists of administering an effective amount of a LM609 grafted antibody or a functional fragment thereof under conditions which allow binding to αᵥβ₃. Also provided is a method of inhibiting a function of ≢ᵥβ₃. The method consists of contacting αᵥβ₃ with a LM609 grafted antibody or a functional fragment thereof under conditions which allow binding to αᵥβ₃.

As described previously, a LM609 grafted antibody is a monoclonal antibody which exhibits essentially all of the binding characteristics as does its parental CDR-donor antibody LM609. These characteristics include, for example, significant binding specificity and affinity for the integrin αᵥβ₃. The Examples below demonstrate these binding properties and further show that the binding of such antibodies to αᵥβ₃ inhibits αᵥβ₃ ligand binding and function. Thus, LM609 grafted antibodies are useful for a large variety of diagnostic and therapeutic purposes directed to the inhibition of αᵥβ₃ function.

The integrin αᵥβ₃ functions in numerous cell adhesion and migration associated events. As such, the dysfunction or dysregulation of this integrin, its function, or of cells expressing this integrin, is associated with a large number of diseases and pathological conditions. The inhibition αᵥβ₃ binding or function can therefore be used to treat or reduce the severity of such αᵥβ₃-mediated pathological conditions. Described below are examples of several pathological conditions mediated by αᵥβ₃ since the inhibition of at least this integrin reduces the severity of the condition. These examples are intended to be representative and as such are not inclusive of all αᵥβ₃-mediated diseases. For example, there are numerous pathological conditions additional to those discussed below which exhibit the dysregulation of βᵥβ₃ binding, function or the dysregulation of cells expressing this integrin and in which the pathological condition can be reduced, or will be found to be reduced, by inhibiting the binding αᵥβ₃. Such pathological conditions which exhibit this criteria, are intended to be included within the definition of the term as used herein.

Angiogenesis, or neovascularization, is the process where new blood vessels form from pre-existing vessels within a tissue. As described further below, this process is mediated by endothelial cells expressing αᵥβ₃ and inhibition of at least this integrin, inhibits new vessel growth. There are a variety of pathological conditions that require new blood vessel formation or tissue neovascularization and inhibition of this process inhibits the pathological condition. As such, pathological conditions that require neovascularization for growth or maintenance are considered to be αᵥβ₃-mediated diseases. The extent of treatment, or reduction in severity, of these diseases will therefore depend on the extent of inhibition of neovascularization. These ≢ᵥβ₃-mediated diseases include, for example, inflammatory disorders such as immune and non-immune inflammation, chronic articular rheumatism, psoriasis, disorders associated with inappropriate or inopportune invasion of vessels such as diabetic retinopathy, neovascular glaucoma and capillary proliferation in atherosclerotic plaques as well as cancer disorders. Such cancer disorders can include, for example, solid tumors, tumor metastasis, angiofibromas, retrolental, fibroplasia, hemangiomas, Kaposi's sarcoma and other cancers which require neovascularization to support tumor growth. Additional diseases which are considered angiogenic include psoriasis and rheumatoid arthritis as well as retinal diseases such as macular degeneration. Diseases other than those requiring new blood vessels which are αᵥβ₃-mediated diseases include, for example, restenosis and osteoporosis.

Treatment of the αᵥβ₃-mediated diseases can be performed by administering an effective amount of a LM609 grafted antibody or a functional fragment thereof so as to bind to αᵥβ₃ and inhibit its function. Administration can be performed using a variety of methods known in the art. The choice of method will depend on the specific αᵥβ₃-mediated disease and can include, for example, the in vivo, in situ and ex vivo administration of a LM609 grafted antibody or functional fragment thereof, to cells, tissues, organs, and organisms. Moreover, such antibodies or functional fragments can be administered to an individual exhibiting or at risk of exhibiting an αᵥβ₃-mediated disease. Definite clinical diagnosis of an αᵥβ₃-mediated disease warrants the administration of a LM609 grafted antibody or a functional fragment thereof. Prophylactic applications are warranted in diseases where the αᵥβ₃-mediated disease mechanisms precede the onset of overt clinical disease. Thus, individuals with familial history of disease and predicted to be at risk by reliable prognostic indicators can be treated prophylactically to interdict αᵥβ₃-mediated mechanisms prior to their onset.

LM609 grafted antibody or functional fragments thereof can be administered in a variety of formulations and pharmaceutically acceptable media for the effective treatment or reduction in the severity of an αᵥβ₃-mediated disease. Such formulations and pharmaceutically acceptable medias are well known to those skilled in the art. Additionally, a LM609 grafted antibody or functional fragments thereof can be administered with other compositions which can enhance or supplement the treatment or reduction in severity of an αᵥβ₃-mediated disease. For example, the coadministration of a LM609 grafted antibody to inhibit tumor-induced neovascularization and a chemotherapeutic drug to directly inhibit tumor growth is one specific case where the administration of other compositions can enhance or supplement the treatment of an αᵥβ₃-mediated disease.

A LM609 grafted antibody or functional fragments are administered by conventional methods, in dosages which are sufficient to cause the inhibition of αᵥβ₃ integrin binding at the sight of the pathology. Inhibition can be measured by a variety of methods known in the art such as in situ immunohistochemistry for the prevalence of αᵥβ₃ containing cells at the site of the pathology as well as include, for example, the observed reduction in the severity of the symptoms of the αᵥβ₃-mediated disease.

In vivo modes of administration can include intraperitoneal, intravenous and subcutaneous administration of a LM609 grafted antibody or a functional fragment thereof. Dosages for antibody therapeutics are known or can be routinely determined by those skilled in the art. For example, such dosages are typically administered so as to achieve a plasma concentration from about 0.01 μg/ml to about 100 μg/ml, preferably about 1-5 μg/ml and more preferably about 5 μg/ml. In terms of amount per body weight, these dosages typically correspond to about 0.1-300 mg/kg, preferably about 0.2-200 mg/kg and more preferably about 0.5-20 mg/kg. Depending on the need, dosages can be administered once or multiple times over the course of the treatment. Generally, the dosage will vary with the age, condition, sex and extent of the αᵥβ₃-mediated pathology of the subject and should not be so high as to cause adverse side effects. Moreover, dosages can also be modulated by the physician during the course of the treatment to either enhance the treatment or reduce the potential development of side effects. Such procedures are known and routinely performed by those skilled in the art.

The specificity and inhibitory activity of LM609 grafted antibodies and functional fragments thereof allow for the therapeutic treatment of numerous αᵥβ₃-mediated diseases. Such diseases include, for example, pathological conditions requiring neovascularization such as tumor growth, and psoriasis as well as those directly mediated by αᵥβ₃ such as restenosis and osteoporosis. Thus, the invention provides methods and LM609 grafted antibody containing compositions for the treatment of such diseases.

Throughout this application various publications are referenced within parentheses. The disclosures of these publications in their entireties are hereby incorporated by reference in this application in order to more fully describe the state of the art to which this invention pertains.

It is understood that modifications which do not substantially affect the activity of the various embodiments of this invention are also included within the definition of the invention provided herein. Accordingly, the following examples are intended to illustrate but not limit the present invention.

### EXAMPLE I

### Isolation and Characterization of LM609 Encoding Nucleic Acids

This Example shows the cloning and sequence determination of LM609 encoding nucleic acids.

LM609 is directed against the human vitronectin receptor, integrin αᵥβ₃. αᵥβ₃ is highly upregulated in melanoma, glioblastoma, and mammary carcinoma and plays a role in the proliferation of M21 melanoma cells both in-vitro and in vivo. αᵥβ₃ also plays a role in angiogenesis, restenosis and the formation of granulation tissue in cutaneous wounds. LM609 has been shown to inhibit the adhesion of M21 cells to vitronectin as well as prevent proliferation of M21 cells in vitro. Thus, grafting of LM609 could result in a clinically valuable therapeutic agent.

cDNA Synthesis of LM609 Variable Regions: For cDNA synthesis, total RNA was prepared from 108 LM609 hybridoma cells using a modification of the method described by Chomczynski and Sacchi (Chomczynski and Sacchi, Analyt. Biochem. 162:156 (1987)). LM609 variable (V) region genes were cloned by reverse transcription-polymerase chain reaction (RT-PCR) and cDNA was synthesized using BRL Superscript kit. Briefly, 5 μg of total cellular RNA, 650 ng oligo dT and H₂O were brought to a total volume of 55 μl. The sample was heated to 70° C. for 10 min and chilled on ice. Reaction buffer was added and the mixture brought to 10 mM DTT and 1 mM dNTPs and heated at 37° C. for 2 minutes. 5 μl (1000 units) reverse transcriptase was added and incubated at 37° C. for 1 hour and then chilled on ice.

All oligonucleotides were synthesized by β-cyanoethyl phosphoramidite chemistry on an ABI 394 DNA synthesizer. Oligonucleotides used for PCR amplification and routine site-directed mutagenesis were purified using oligonucleotide purification cartridges (Applied Biosystems, Foster City, Calif.). Forward PCR primers were designed from N-terminal protein sequence data generated from purified LM609 antibody. The forward PCR primers contained sequences coding for the first six amino acids in each antibody variable chain (protein sequenced at San Diego State University). The sequence of the light chain forward PCR primer (997) was 5′-GCC CAA CCA GCC ATG GCC GAT ATT GTG CTA ACT CAG-3′ (SEQ ID NO:19) whereas the light chain reverse PCR primer (734) was 5′-AC AGT TGG TGC AGC ATC AGC-3′ (SEQ ID NO:20) used. This reverse primer corresponds to mouse light chain kappa amino acid residues 109-115. The sequence of the heavy chain forward PCR primer (998) was 5′-ACC CCT GTG GCA AAA GCC GAA GTG CAG CTG GTG GAG-3′ (SEQ ID NO:21). Heavy chain reverse PCR primer 733: 5′-GA TGG GGG TGT CGT TTT GGC-3′ SEQ ID NO:22). The PCR primers also contain regions of homology with specific sequences within the immunoexpression vector.

V_{L} and V_{H} chains were amplified in two separate 50 μl reaction mixtures containing 2 μl of the cDNA-RNA heteroduplex, 66.6 mM Tris-HCl pH 8.8, 1.5 MM MgCl₂, 0.2 mM of each four dNTPs, 10 mM 2-mercaptoethanol, 0.25 units Taq polymerase (Boehringer-Mannheim, Indianapolis, Ind.) and 50 pmoles each of primers 997 and 734 and 998 and 733, respectively. The mixtures were overlaid with mineral oil and cycled for two rounds of PCR with each cycle consisting of 30 seconds at 94° C. (denature), 30 seconds at 50° C. (anneal), and 30 seconds at 72° C. (synthesis). This reaction was immediately followed by 0.30 cycles of PCR consisting of 30 seconds at 94° C. (denature), 30 seconds at 55° C. (anneal), and 30 seconds at 72° C. (synthesis) followed by a final synthesis reaction for 5 minutes at 72° C. The reaction products were pooled, extracted with CHCl₃ and ethanol precipitated.

Amplified products were resuspended in 20 μl TE buffer (10 mM Tris-HCl, 1 mM EDTA, pH 8.0) and electrophoresed on a 5% polyacrylamide gel. Bands migrating at expected molecular weights of V_{H} and V_{L} were excised, chemically eluted from the gel slice, extracted with organic solvents and ethanol precipitated.

Cloning of amplified V_{H} and V_{L} genes into M13 phage immunoexpression vector: The amplified V region gene products were sequentially cloned into the phage immunoexpression vector by hybridization mutagenesis (Near, R. Biotechniques 12:88 (1992); Yelton et al., J. Immunol. 155:1994-2003 (1995)). Introduction of the amplified V_{L} and V_{H} sequences by hybridization mutagenesis positions the antibody sequences in frame with the regulatory elements contained in the M13 vector required for efficient Fab expression. One advantage of this technique is that no restriction endonuclease sites need to be incorporated into the V_{L} or V_{H} gene sequences for cloning as is done with conventional DNA ligation methods.

To perform the cloning, 400 ng each of the double-stranded amplified products were first phosphorylated with polynucleotide kinase. 100 ng of the phosphorylated LM609 V_{L} product was mixed with 250 ng of uridinylated BS11 phage immunoexpression vector, denatured by heating to 90° C. and annealed by gradual cooling to room temperature. BS11 is an M13 immunoexpression vector derived from M13 IX and encodes CH₁ of murine IgG1 and murine kappa light chain constant domain (Huse, W. D. In: Antibody Engineering: A Practical Guide, C. A. K. Borrebaeck, ed. W.H. Freeman and Co., Publishers, New York, pp. 103-120 (1991)). Nucleotide sequences included in the PCR amplification primers anneal to complementary sequences present in the single-stranded BS11 vector. The annealed mixture was fully converted to a double-stranded molecule with T4 DNA polymerase plus dNTPs and ligated with T4 ligase. 1 μl of the mutagenesis reaction was electroporated into E. coli strain DH10B, titered onto a lawn of XL-1 E. coli and incubated until plaques formed. Plaque lift assays were performed as described using goat anti-murine kappa chain antibody conjugated to alkaline phosphatase (Yelton et al, supra; Huse, W. D., supra). Fifteen murine light chain positive M13 phage clones were isolated, pooled and used to prepare uridinylated vector to serve as template for hybridization mutagenesis with the PCR amplified LM609 V_{H} product.

Clones expressing functional murine LM609 Fab were identified by binding to purified αᵥβ₃ by ELISA. Briefly, Immulon II ELISA plates were coated overnight with 1 μg/ml (100 ng/well) αᵥβ₃ and nonspecific sites blocked for two hours at 27° C. Soluble Fabs were prepared by isolating periplasmic fractions of cultures of E. coli strain MK30-3 (Boehringer Mannheim Co.) infected with the Fab expressing M13 phage clones. Periplasm fractions were mixed with binding buffer 100 mM NaCl, 50 mM Tris pH 7.4, 2 mM CaCl₂₁ 1 mM MgCl₂, 1 mM MnCl₂₁ 0.02% NaN₃, 1 mg/ml BSA and incubated with immobilized αᵥβ₃ for two hours at 27° C. Plates were washed with binding buffer and bound Fab detected with goat anti-murine kappa chain antibody conjugated to alkaline phosphatase. Four αᵥβ₃ reactive clones were identified: muLM609M13 12, 29, 31 and 69. MuLM609M13 12 and 29 gave the strongest signals in the ELISA assay. DNA sequence analysis showed that clones muLM609M13 12, 31 and 69 all had identical light chain sequence and confirmed the previously determined N-terminal amino acid sequence of purified LM609 light chain polypeptide. All four clones had identical V_{H} DNA sequence and also confirmed the previously determined N-terminal amino acid sequence of purified LM609 heavy chain polypeptide.

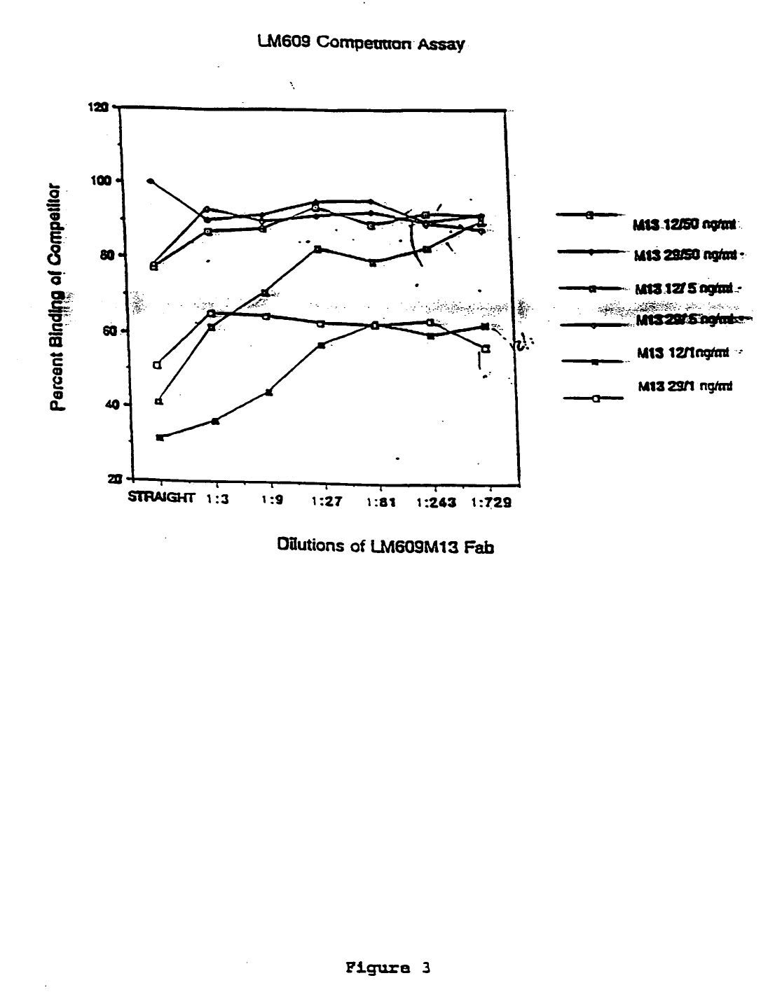

To further characterize the binding activity of each clone, soluble Fab fractions were prepared from 50 ml cultures of E. coli strain MK30-3 infected with clones 12 and 29 and evaluated for binding to αᵥβ₃ in a competitive ELISA with LM609 IgG. The results of this ELISA are shown in FIG. 3. Clone muLM609M13 12 was found to inhibit LM609 IgG binding (at LM609 IgG concentrations of 1 ng/ml and 5 ng/ml) to α₁β₃ in a concentration dependent manner at periplasm titers ranging from neat to 1:80. Clone muLM609M13 12 was plaque purified and both the V region heavy and light chain DNA sequences again determined. Complete DNA sequence of the final clone, muLM609M13 12-5, is shown in FIGS. 2A and 2B.

### EXAMPLE II

### Construction of LM609 Grafted Functional Antibody Fragments

This Example shows the construction of functional LM609 grafted antibody fragments in which only the CDRs have been transferred from the LM609 donor antibody to a human acceptor framework.

CDR grafting of LM609 to produce a functional antibody fragment was accomplished by the methods set forth below. These procedures are applicable for the CDR grafting of essentially any donor antibody where amino acid residues outside of the CDRs from the donor antibody are not desired in the final grafted product.

Briefly, the protein sequence of the LM609 antibody, was determined by cloning and sequencing the cDNA that encodes the variable regions of the heavy and light chains as described in Example I. The CDRs from the LM609 donor antibody were identified and grafted into homologous human variable regions of a human acceptor framework. Identification of CDR regions were based on the combination of definitions published by Kabat et al., and MacCallum et al.

The boundaries of the CDR regions have been cumulatively defined by the above two publications and are residues 30-35, 47-66 and 97-106 for CDRs 1, 2 and 3, respectively, of the heavy chain variable region and residues 24-36, 46-56, and 89-97 for CDRs 1, 2 and 3, respectively, of the light chain variable region. Non-identical donor residues within these boundaries but outside of CDRs as defined by Kabat et al. were identified and were not substituted into the acceptor framework. Instead, functional non-donor amino acid residues were identified and substituted for certain of these non-identical residues.

As described below, the only non-identical residue outside of the CDRs as defined by Kabat et al. but within the CDRs as defined above is at position 49 of the LM609 light chain. To identify functional non-donor amino acids at this position, a library of nineteen antibodies was constructed that contained all non-donor amino acids at position 49 and then screened for binding activity against αᵥβ₃.

Human immunoglobulin sequences were identified from the Brookhaven Protein Data Bank-Kabat Sequences of Proteins of Immunological Interest database (release 5.0). Human framework sequences showing significant identity to the murine LM609 variable region gene sequences were selected for receiving the LM609 CDRs. Human heavy chain variable region M72 'CL had 88% identity to frameworks 1, 2 and 3 of LM609 heavy chain and human light chain V region LS1 'CL had 79% identity to frameworks 1, 2 and 3 of LM609 light chain. With the exclusion of non-identical residues outside of the CDRs as defined by Kabat et al. murine LM609 CDR sequences as defined by Kabat et al. and MacCallum et al. were grafted onto the human frameworks. Using this grafting scheme, the final grafted product does not contain any amino acid residues outside of the CDRs as defined by Kabat et al. which are identical to an LM609 amino acid at the corresponding position (outside of residues: 31-35, 50-66 and 99-106 for CDRs 1, 2 and 3, respectively, of the heavy chain variable region and residues 24-34, 50-56, and 89-97 for CDRs 1, 2 and 3, respectively, of the light chain variable region). Moreover, no intermediates are produced which contain an amino acid residue outside of the CDRs as defined by Kabat et al. which are identical to the LM609 amino acid at that position. The CDR grafting procedures are set forth below.

Full-length CDR grafted variable region genes were synthesized by PCR using long overlapping oligonucleotides. The heavy chain oligonucleotides map to the following nucleotide positions: V_{H} oligonucleotide 1 (V_{H} oligo1), nucleotides (nt) 1-84; (SEQ ID NO:9); V_{H} oligo2, nt 70-153, (SEQ ID NO:10); V_{H} oligo3, nt 138-225 (SEQ ID NO:11); V_{H} oligo4, nt 211-291 (SEQ ID NO:12); V_{H} oligo5, nt 277-351 (SEQ ID NO:13).

The light chain variable region oligonucleotides were synthesized so as to contain the CDR grafted variable region as well as a stop condon at position 49. The five oligonucleotides for the light chain LM609 grafted variable region are shown as SEQ ID NOS:14-18 where the second oligonucleotide in the series contains the stop codon at position 49 (SEQ ID NO:15).

All long oligonucleotides were gel purified. CDR grafting of the LM609 heavy chain variable region was constructed by mixing 5 overlapping oligonucleotides (SEQ ID NOS:9-13), at equimolar concentrations, in the presence of annealing PCR primers containing at least 18 nucleotide residues complementary to vector sequences for the efficient annealing of the amplified V region product to the single-stranded vector. The annealed mixture was fully converted to a double-stranded molecule with T4 DNA polymerase plus dNTPs and ligated with T4 ligase. The mutagenesis reaction (1 μl) was electroporated into E. coli strain DH10B (BRL), titered onto a lawn of XL-1 (Stratagene, Inc.) and incubated until plaques formed. Replica filter lifts were prepared and plaques containing V_{H} gene sequences were screened either by hybridization with a digoxigenin-labeled oligonucleotide complementary to LM609 heavy chain CDR 2 sequences or reactivity with 7F11-alkaline phosphatase conjugate, a monoclonal antibody raised against the decapeptide sequence Tyr Pro Tyr Asp Val Pro Asp Tyr Ala Ser (SEQ ID NO:23) appended to the carboxy terminus of the vector CH₁ domain (Biosite, Inc., San Diego, Calif.).

Fifty clones that were double-positive were pooled and used to prepare uridinylated template for hybridization mutagenesis with the amplified CDR grafted LM609 V_{L} product constructed in a similar fashion using the five overlapping oligonucleotides shown as SEQ ID NOS:23-27. The mutagenesis reaction was electroporated into E. coli strain DH10B. Randomly picked clones were sequenced to identify a properly constructed template for construction of the non-donor library at position 49. This template was prepared as a uridinylated template and an oligonucleotide population of the following sequence was used for site directed mutagenesis.

GGGAACGATA-19aa-GATGAGAAGC

The sequence 19aa in the above primer (SEQ ID NO:24) represents the fact that this primer specifies a sequence population consisting of 19 different codon sequences that encode each of the 19 non-donor amino acids. These amino acids are those not found at position 49 of LM609 and include all amino acids except for Lys. Clones that resulted from this mutagenesis were picked and antibody expressed by these clones were prepared. These samples were then screened for binding to αᵥβ₃ in an ELISA assay. Clones having either Arg or Met amino acids in position 49 were functionally identified. The nucleotide and amino acid sequence of the LM609 grafted heavy chain variable region is show in FIG. 1A (SEQ ID NOS:1 and 2, respectively). The nucleotide and amino acid sequence of the LM609 grafted light chain variable region is shown in FIG. 1B (SEQ ID NOS:3 and 4, respectively).

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1. A LM609 grafted antibody exhibiting selective binding affinity to αᵥβ₃ comprising at least one LM609 grafted heavy chain polypeptide comprising substantially the same variable region amino acid sequence as that shown in FIG. 1A (SEQ ID NO:2) and at least one LM609 grafted light chain polypeptide comprising substantially the same variable region amino acid sequence as that shown in FIG. 1B (SEQ ID NO:4) or a functional fragment thereof.

2. The LM609 grafted antibody of claim 1, wherein said functional fragment is selected from the group consisting of Fv, Fab, F(ab)₂ and scFV.

3. A nucleic acid encoding a LM609 grafted heavy chain polypeptide comprising substantially the same LM609 grafted heavy chain variable region nucleotide sequences as that shown in FIG. 1A (SEQ ID NO:1) or a fragment thereof.

4. The nucleic acid of claim 3, wherein said fragment further comprises a nucleic acid encoding substantially the same nucleotide sequence as the variable region of said LM609 grafted heavy chain polypeptide (SEQ ID NO:1).

5. The nucleic acid of claim 3, wherein said fragment further comprises a nucleic acid encoding substantially the same nucleotide sequence as a CDR of said LM609 grafted heavy chain polypeptide.

6. A nucleic acid encoding a LM609 grafted light chain polypeptide comprising substantially the same LM609 grafted light chain variable region nucleotide sequences as that shown in FIG. 1B (SEQ ID NO:3) or a fragment thereof.

7. The nucleic acid of claim 6, wherein said fragment further comprises a nucleic acid encoding substantially the same nucleotide sequence as the variable region of said LM609 grafted light chain polypeptide (SEQ ID NO:3).

8. The nucleic acid of claim 6, wherein said fragment further comprises a nucleic acid encoding substantially the same nucleotide sequence as a CDR of said LM609 grafted light chain polypeptide.

9. A nucleic acid encoding a LM609 grafted antibody heavy chain polypeptide comprising a nucleotide sequence encoding substantially the same LM609 grafted heavy chain variable region amino acid sequence as that shown in FIG. 1A (SEQ ID NO:2) or fragment thereof.

10. The nucleic acid of claim 9, wherein said fragment further comprises a nucleic acid encoding substantially the same heavy chain variable region amino acid sequence of said LM609 grafted heavy chain amino acid sequence (SEQ ID NO:2).

11. The nucleic acid of claim 9, wherein said fragment further comprises a nucleic acid encoding substantially the same heavy chain CDR amino acid sequence of said LM609 grafted heavy chain amino acid sequence.

12. A nucleic acid encoding a LM609 grafted antibody light chain polypeptide comprising a nucleotide sequence encoding substantially the same LM609 grafted light chain variable region amino acid sequence as that shown in FIG. 1B (SEQ ID NO:4) or fragment thereof.

13. The nucleic acid of claim 12, wherein said fragment further comprises a nucleic acid encoding substantially the same light chain variable region amino acid sequence of said LM609 grafted light chain amino acid sequence (SEQ ID NO:4).

14. The nucleic acid of claim 12, wherein said fragment further comprises a nucleic acid encoding substantially the same light chain CDR amino acid sequence of said LM609 grafted light chain amino acid sequence.

15. A LM609 grafted heavy chain polypeptide comprising substantially the same variable region amino acid sequence as that shown in FIG. 1A (SEQ ID NO:2) or functional fragment thereof.

16. The LM609 grafted heavy chain polypeptide of claim 15, wherein said functional fragment comprises a variable chain polypeptide or a CDR polypeptide.

17. A LM609 grafted light chain polypeptide comprising substantially the same variable region amino acid sequence as that shown in FIG. 7 (SEQ ID NO:4) or a functional fragment thereof.

18. The LM609 grafted light chain polypeptide of claim 17, wherein said functional fragment comprises a variable chain polypeptide or a CDR polypeptide.

19. A method of inhibiting a function of αᵥβ₃ comprising contacting αᵥβ₃ with a LM609 grafted antibody or a functional fragment thereof under conditions which allow binding of LM609 grafted antibodies to αᵥβ₃.

20. The method of claim 19, wherein said functional fragment is selected from the group consisting of Fv, Fab, F(ab)₂ and scFV.

21. The method of claim 19, wherein said function of αᵥβ₃ is binding of αᵥβ₃ to a ligand.

22. The method of claim 19, wherein said function of αᵥβ₃ is integrin mediated signal transduction.

23. A method of treating an αᵥβ₃-mediated disease comprising administering an effective amount of a LM609 grafted antibody or a functional fragment thereof under conditions which allow binding to αᵥβ₃.

24. The method of claim 23, wherein said functional fragment is selected from the group consisting of Fv, Fab, F(ab)₂ and scFV.

25. The method of claim 23, wherein said αᵥβ₃-mediated disease is angiogenesis or restenosis.
